(19) United States
(12) Patent Application Publication
(10) Pub. No.: US 2005/0233980 A1
(43) Pub. Date: Oct. 20, 2005

(54) CGRP-antagonist in combination with a serotonin-reuptake inhibitor for the treatment of migraine

(75) Inventors: Henri Doods, Warthausen (DE); Klaus Rudolf, Warthausen (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 11/108,985

(22) Filed: Apr. 19, 2005

Related U.S. Application Data
(60) Provisional application No. 60/570,379, filed on May 12, 2004.

(30) Foreign Application Priority Data
Apr. 20, 2004 (DE) 10 2004 019 736
Dec. 29, 2004 (DE) 10 2004 063 754

(51) Int. Cl. A61K 38/04

(52) U.S. Cl. 514/19

(57) ABSTRACT

The present invention relates to a process for the treatment or prevention of headaches, migraine or cluster headache, this process comprising the joint administration of a therapeutically effective amount of the CGRP-antagonist (A) or a physiologically acceptable salt thereof and a therapeutically effective amount of the selective serotonin reuptake inhibitor (B) or a physiologically acceptable salt thereof, as well as the corresponding pharmaceutical compositions and the preparation thereof.

### BACKGROUND TO THE INVENTION

Migraine is one of the most common neurological disorders and comprises periodic attacks of headache and nausea and a variety of other symptoms. Although considerable progress has been made, the pathophysiology of migraine is far from understood. A number of observations have pointed to the involvement of the “calcitonin gene related peptide” (CGRP). Migraine headaches involve the activation of the trigeminal system and the dilation of cranial blood vessels. CGRP is located in the neurons in trigeminal ganglia, and the CGRP levels are raised during a migraine attack, which is presumably what causes the vasodilatation observed. It is therefore conceivable that inhibiting the dilation of the cranial blood vessels caused by CGRP might possibly give rise to a new treatment for migraine headaches.

Medicaments widely used for treating migraine are the so-called “triptans”, e.g. sumatriptan and zolmitriptan. These compounds derive their activity against migraine from their vasoconstrictor properties and presumably their inhibition of the release of the neuropeptide “calcitonin gene related peptide” (CGRP) (Ferrari, M. D., Saxena, P. R. (1995), 5-HT1 receptors in migraine pathophysiology and treatment, Eur. J. Neurology, 2, 5-21; Johnson, K. W., Phebus, L. A., Cohen, M. L. (1998), Serotonin in migraine: Theories, animal models and emerging therapies, Progress in Drug Research, vol. 51, 220-244), assuming that the levels thereof are raised during a migraine attack (Edvinsson, L., Goadsby, P. J. (1994), Neuropeptides in migraine and cluster headache, Cephalgia, 14(5), 320-327). A completely new approach for the treatment of migraine is the use of CGRP antagonists (Doods, H., Hallermayer, G., Wu, D., Entzeroth, M., Rudolf, K., Engel, W., Eberlein, W. (2000), Pharmacological profile of BIBN4096BS, the first selective small molecule CGRP antagonist, Br. J. Pharmacol., 129, 420-423).

### BACKGROUND TO THE INVENTION

WO 98/11128 discloses modified amino acids with CGRP-antagonistic properties, the use thereof and processes for the preparation thereof as well as the use thereof for the preparation and purification of antibodies and as labelled compounds in RIA and ELISA assays and as diagnostic or analytical aids in neurotransmitter research. In view of their pharmacological properties the modified amino acids are therefore suitable for the acute and prophylactic treatment of headaches, particularly migraine and cluster headaches.

### SUMMARY OF THE INVENTION

Surprisingly it was found that in a model assumed to predict the anti-migraine activities of pharmaceutical compositions, the combination of two pharmaceutical compositions with completely different modes of activity, namely the CGRP antagonist 1-[N²-[3,5-dibromo-N-[[4-(3,4-dihydro-2(1H)-oxoquinazolin-3-yl)-1-piperidinyl]-carbonyl]-D-tyrosyl]-L-lysyl]4-(4-pyridinyl)-piperazine 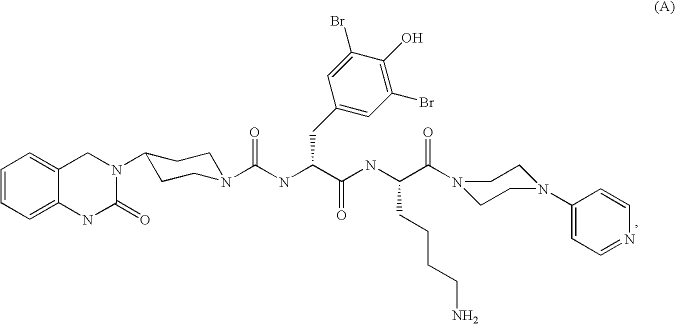
or a physiologically acceptable salt thereof and the selective serotonin reuptake inhibitor (+)-N-methyl-3-(1-naphthyloxy)-3-(2-thienyl)propanamine 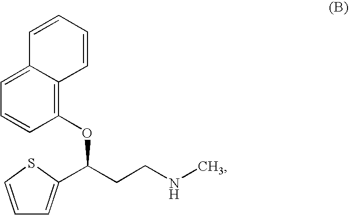
or a physiologically acceptable salt thereof leads to an improved activity compared with the activity of only one pharmaceutical composition.

### DETAILED DESCRIPTION OF THE INVENTION

In a first aspect the present invention relates to a process for the treatment or prevention of indications which are selected from among the group comprising headaches, migraine and cluster headaches, this process comprising the simultaneous administration of a therapeutically effective amount of the CGRP-antagonist (A) or a physiologically acceptable salt thereof and a therapeutically effective amount of the selective serotonin reuptake inhibitor (B) or a physiologically acceptable salt thereof to a person in need of such treatment.

The dosage for the selective serotonin reuptake inhibitor (B) is roughly 1/50 of the lowest normally recommended dose to 1/1 of the normally recommended dose, by oral, nasal, inhalative, subcutaneous or intravenous route.

According to the invention the CGRP antagonist (A) or a physiologically acceptable salt thereof may be administered by intravenous or subcutaneous route in a dosage of 0.0001 to 3 mg/kg of body weight, by oral route in a dosage of 0.1 to 20 mg/kg body weight or by nasal or inhalative route in a dosage of 0.1 to 10 mg/kg body weight once, twice or three times a day, in combination with
the selective serotonin reuptake inhibitor (B) or a physiologically acceptable salt thereof, which may be administered by oral route in a dosage of 0.03 to 1.43 mg/kg body weight once, twice or three times a day or
by intravenous or subcutaneous route in a dosage of 0.002 to 0.09 mg/kg body weight once or twice a day or
by rectal route in a dosage of 0.007 to 0.36 mg/kg body weight once or twice a day or
by nasal route in a dosage of 0.006 to 0.29 mg/kg body weight once or twice a day.

In a second aspect the present invention provides a pharmaceutical composition for the treatment or prevention of headaches. migraine or cluster headache, which consists of a therapeutically effective amount of the CGRP-antagonist (A) or a physiologically acceptable salt thereof and the selective serotonin reuptake inhibitor (B) or a physiologically acceptable salt thereof, as a combined preparation for simultaneous or sequential administration.

A pharmaceutical composition according to the invention may contain a single dosage unit of 0.1 to 1500 mg, preferably 0.3 to 1000 mg, particularly preferably 5 to 750 mg, of the CGRP-antagonist (A) or an equivalent amount of a physiologically acceptable salt thereof and
a single dosage unit of 0.1 to 150 mg, preferably 0.2 to 100 mg, for example 10 to 100 mg, particularly preferably 10 to 80 mg, particularly 40 to 80 mg, of the selective serotonin reuptake inhibitor (B) or an equivalent amount of a physiologically acceptable salt thereof.

All the doses or dosage units of a physiologically acceptable salt of one of the above-mentioned active compounds should be understood as being doses or dosages of the active compound itself.

Moreover a pharmaceutical composition according to the invention may be a kit of parts for the treatment or prevention of headache, migraine or cluster headaches, the kit comprising:
(a) a first enclosure containing a pharmaceutical composition comprising a therapeutically effective amount of the CGRP antagonist (A) or a physiologically acceptable salt thereof and one or more physiologically acceptable diluents and/or carriers; and
(b) a second enclosure containing a pharmaceutical composition comprising the selective serotonin reuptake inhibitor (B) or a physiologically acceptable salt thereof and one or more physiologically acceptable diluents and/or carriers.

In a third aspect the present invention relates to the use of the CGRP antagonist (A) or a physiologically acceptable salt thereof in combination with the selective serotonin reuptake inhibitor (B) or a physiologically acceptable salt thereof for preparing a pharmaceutical composition for the treatment or prevention of headaches, migraine or cluster headache.

The CGRP antagonist (A) or a physiologically acceptable salt thereof may be administered e.g. using one of the pharmaceutical formulations described in the Examples. The Examples that follow describe pharmaceutical compositions which contain the CGRP antagonist (A) or a physiologically acceptable salt thereof and the selective serotonin reuptake inhibitor (B) or a physiologically acceptable salt thereof.

### EXAMPLE 1a

Tablets Containing 100 mg CGRP Antagonist (A) or an Equivalent Amount of a Physiologically Acceptable Salt Thereof and 50 mg Serotonin Reuptake Inhibitor (B) or an Equivalent Amount of a Physiologically Acceptable Salt Thereof

Composition/Tablet:

| | | |
|---|---|---|
| CGRP antagonist (A) | 100 | mg |
| serotonin reuptake inhibitor (B) | 50 | mg |
| lactose | 375 | mg |
| magnesium stearate | 3.0 | mg |
| povidone | 8.5 | mg |
| crospovidone | 14.4 | mg |

volatile component: water

Method of Preparation:

CGRP antagonist (A), serotonin reuptake inhibitor (B) and lactose (fine) are homogeneously mixed in a suitable mixer (e.g. Diosna P2); then the mixture is granulated with an aqueous povidone solution. The granulated material is screened through a 1.6 mm Kressner screen and dried for 2 hours at 40° C. Then the granules are screened in a suitable mill, e.g. a Comill, with a mesh size of 1.1 mm, at 3000 rpm. The granules are then mixed with crospovidone for 5 minutes and then with magnesium stearate for 1 minute. The mixture thus obtained is compressed in a tablet press to produce tablets of suitable diameter.

This method is the basis for other examples listed in the Table that follows.

TABLE 1

| | CGRP | serotonin | | | | |
|---|---|---|---|---|---|---|
| | antagonist | reuptake inhibitor | mg | mg | mg | mg Mg- |
| Example | (A) [mg] | (B) [mg] | lactose | povidone | crospovidone | stearate |
| 1.1 | 570 | 10 | 1160.0 | 26.1 | 44.2 | 9.1 |
| 1.2 | 430 | 50 | 960.0 | 21.6 | 36.5 | 7.5 |
| 1.3 | 560 | 20 | 470.0 | 26.1 | 44.2 | 9.1 |
| 1.4 | 560 | 50 | 630.0 | 18.6 | 31.5 | 6.5 |
| 1.5 | 480 | 50 | 820.0 | 20.3 | 34.3 | 7.0 |
| 1.6 | 430 | 70 | 645.0 | 17.2 | 29.1 | 6.0 |
| 1.7 | 250 | 40 | 580.0 | 13.1 | 22.1 | 4.5 |
| 1.8 | 510 | 20 | 870.0 | 21.0 | 35.5 | 7.3 |
| 1.9 | 240 | 70 | 620.0 | 14.0 | 23.6 | 4.8 |
| 1.10 | 310 | 60 | 740.0 | 16.7 | 28.2 | 5.8 |
| 1.11 | 190 | 70 | 520.0 | 11.7 | 19.8 | 4.1 |
| 1.12 | 10 | 10 | 140.0 | 2.4 | 4.1 | 0.8 |
| 1.13 | 540 | 70 | 790.0 | 21.0 | 35.5 | 7.3 |
| 1.14 | 1000 | 10 | 40.0 | 15.8 | 26.6 | 5.5 |
| 1.15 | 390 | 40 | 860.0 | 19.4 | 32.7 | 6.7 |
| 1.16 | 350 | 70 | 840.0 | 18.9 | 32.0 | 6.6 |
| 1.17 | 240 | 40 | 560.0 | 12.6 | 21.3 | 4.4 |
| 1.18 | 250 | 40 | 580.0 | 13.1 | 22.1 | 4.5 |
| 1.19 | 40 | 40 | 160.0 | 3.6 | 6.1 | 1.2 |
| 1.20 | 270 | 50 | 640.0 | 14.4 | 24.4 | 5.0 |
| 1.21 | 460 | 60 | 660.0 | 17.7 | 29.9 | 6.1 |
| 1.22 | 490 | 70 | 540.0 | 16.5 | 27.9 | 5.7 |
| 1.23 | 150 | 60 | 420.0 | 9.5 | 16.0 | 3.3 |
| 1.24 | 20 | 20 | 80.0 | 1.8 | 3.0 | 0.6 |
| 1.25 | 40 | 10 | 100.0 | 2.3 | 3.8 | 0.8 |

### EXAMPLE 2

Tablets Containing 100 mg CGRP Antagonist (A) or an Equivalent Amount of a Physiologically Acceptable Salt Thereof and 50 mg Serotonin Reuptake Inhibitor (B) or an Equivalent Amount of a Physiologically Acceptable Salt Thereof

Composition:

| | | |
|---|---|---|
| CGRP antagonist (A) | 100 | mg |
| serotonin reuptake inhibitor (B) | 50 | mg |
| lactose | 284 | mg |
| microcrystalline cellulose | 89.5 | mg |
| magnesium stearate | 7.2 | mg |
| croscarmellose | 7.3 | mg |

volatile component: water

Method of Preparation:

CGRP antagonist (A), serotonin reuptake inhibitor (B), lactose (fine) and microcrystalline cellulose are homogeneously mixed in a suitable mixer (e.g. Diosna P2); then the mixture is granulated with water. The granulated material is screened through a 1.6 mm Kressner screen and dried for 2 hours at 40° C. Then the granules are screened in a suitable mill, e.g. a Comill, at 3000 rpm with a mesh size of 1.1 mm. The granules are then mixed with croscarmellose for 5 minutes and then with magnesium stearate for 1 minute. The mixture thus obtained is compressed in a tablet press to produce tablets of suitable diameter.

This method is the basis for other examples listed in the Table that follows.

TABLE 2

| | | serotonin | | mg | mg | |
|---|---|---|---|---|---|---|
| | CGRP antagonist | reuptake inhibitor | mg | microcryst. | Mg- | mg cros- |
| Example | (A) [mg] | (B) [mg] | lactose | cellulose | stearate | carmellose |
| 2.1 | 210 | 80 | 435.0 | 145.0 | 13.1 | 13.2 |
| 2.2 | 10 | 100 | 165.0 | 55.0 | 5.0 | 5.0 |
| 2.3 | 80 | 10 | 135.0 | 45.0 | 4.1 | 4.1 |
| 2.4 | 210 | 70 | 420.0 | 140.0 | 12.6 | 12.8 |
| 2.5 | 260 | 20 | 420.0 | 140.0 | 12.6 | 12.8 |
| 2.6 | 140 | 20 | 240.0 | 80.0 | 7.2 | 7.3 |
| 2.7 | 10 | 60 | 105.0 | 35.0 | 3.2 | 3.2 |
| 2.8 | 10 | 30 | 60.0 | 20.0 | 1.8 | 1.8 |
| 2.9 | 280 | 60 | 380.0 | 144.0 | 13.0 | 13.2 |
| 2.10 | 120 | 60 | 270.0 | 90.0 | 8.1 | 8.2 |
| 2.11 | 80 | 20 | 150.0 | 50.0 | 4.5 | 4.6 |
| 2.12 | 240 | 60 | 450.0 | 150.0 | 13.5 | 13.7 |
| 2.13 | 290 | 30 | 480.0 | 160.0 | 14.4 | 14.6 |
| 2.14 | 320 | 100 | 360.0 | 156.0 | 14.0 | 14.3 |
| 2.15 | 80 | 60 | 210.0 | 70.0 | 6.3 | 6.4 |
| 2.16 | 190 | 50 | 360.0 | 120.0 | 10.8 | 11.0 |
| 2.17 | 60 | 40 | 150.0 | 50.0 | 4.5 | 4.6 |
| 2.18 | 10 | 10 | 30.0 | 10.0 | 0.9 | 0.9 |
| 2.19 | 20 | 10 | 45.0 | 15.0 | 1.4 | 1.4 |
| 2.20 | 360 | 50 | 370.0 | 156.0 | 14.0 | 14.3 |

### EXAMPLE 3a

Aqueous Solution for Intranasal Application Containing 20% CGRP Antagonist (A) or an Equivalent Amount of a Physiologically Acceptable Salt Thereof and 5% Serotonin Reuptake Inhibitor (B) or an Equivalent Amount of a Physiologically Acceptable Salt Thereof

Composition:

| | | |
|---|---|---|
| CGRP antagonist (A) | 20 | mg |
| serotonin reuptake inhibitor (B) | 5 | mg |
| mannitol | 5 | mg |
| water | ad 0.1 | ml |

Method:

The active substance is dissolved/suspended in water with stirring and optionally heating. The isotonic agent mannitol is added and the solution is made up to the final volume with water.

### EXAMPLE 3b

Aqueous Solution for Intranasal Application Containing 40% CGRP Antagonist (A) or an Equivalent Amount of a Physiologically Acceptable Salt Thereof and 10% Serotonin Reuptake Inhibitor (B) or an Equivalent Amount of a Physiologically Acceptable Salt Thereof and 1.5% Labrasol

Composition:

| | | |
|---|---|---|
| CGRP antagonist (A) | 40 | mg |
| serotonin reuptake inhibitor (B) | 10 | mg |
| Labrasol | 1.5 | mg |
| mannitol | 5 | mg |
| water | ad 0.1 | ml |

Method:

The active substances are dissolved/suspended in water with stirring and optionally heating. The isotonic agent mannitol and Labrasol are added and the solution is made up to the final volume with water.

This method is the basis for other Examples which are listed in the following Table.

Table Relating to 3a and b

| | | serotonin | | | |
|---|---|---|---|---|---|
| | CGRP | reuptake | | | |
| | antagonist | inhibitor | mg | mg | |
| Example | (A) [mg] | (B) [mg] | Mannitol | Labrasol | mg water |
| 3.1 | 20 | 3.5 | 5 | 3.00 | 68.50 |
| 3.2 | 70 | 4.8 | 5 | 3.00 | 17.16 |
| 3.3 | 50 | 2.5 | 5 | 1.50 | 41.00 |
| 3.4 | 40 | 2.8 | 5 | 0.00 | 52.22 |
| 3.5 | 30 | 5.0 | 5 | 0.00 | 60.00 |
| 3.6 | 60 | 2.0 | 5 | 3.00 | 30.00 |
| 3.7 | 20 | 10.0 | 5 | 1.50 | 63.50 |
| 3.8 | 30 | 5.0 | 5 | 1.50 | 58.50 |
| 3.9 | 10 | 4.0 | 5 | 0.00 | 81.00 |
| 3.10 | 70 | 10.0 | 5 | 0.00 | 15.00 |
| 3.11 | 70 | 4.0 | 5 | 3.00 | 18.00 |
| 3.12 | 60 | 2.5 | 5 | 3.00 | 29.50 |
| 3.13 | 5 | 4.0 | 5 | 3.00 | 83.00 |
| 3.14 | 30 | 20.0 | 5 | 3.00 | 42.00 |
| 3.15 | 70 | 5.0 | 5 | 1.50 | 18.50 |

Pellets

The medicaments according to the invention may also be prepared in the form of small particles such as e.g. pellets. The active substance may be applied to neutral pellets consisting of sucrose and starch or microcrystalline cellulose.

The preparation comprises the following steps:
1. selection or preparation of starter pellets
2. build-up of the layer of active substance
Optional: coating the pellets to improve their stability or correct the flavour or—if desired—to delay the release of one or more active substances.

### EXAMPLE 4

Preparation of an Application of Active Substance Comprising 100 Parts by Weight CGRP Antagonist (A) or an Equivalent Amount of a Physiologically Acceptable Salt Thereof and 40 Parts by Weight Serotonin Reuptake Inhibitor (B) or an Equivalent Amount of a Physiologically Acceptable Salt Thereof

Composition:

| | |
|---|---|
| core material | 200 parts by weight |
| hydroxypropylcellulose | 38 parts by weight |
| talc | 20 parts by weight |
| CGRP antagonist (A) | 100 parts by weight |
| serotonin reuptake inhibitor (B) | 40 parts by weight |

Hydroxypropylcellulose is dissolved in 250 parts by weight of 2-propanol with stirring and then the active substances and talc are dispersed in this solution with stirring. In a fluidised bed processor 200 parts by weight of core material are sprayed with the dispersion containing the active substance at an air inlet temperature of 20° C. to 30° C. using the under-bed spraying method. The pellets containing the active substance are then dried in the circulating air dryer at 35° C. for 8 hours.

To remove lumps the pellets containing the active substance are screened through a screen with a nominal mesh size of 1.25 mm. The product fraction (particle size<1.25 mm) is processed further.

The layer of active substance is generally always produced in the same way, but the nature and amount of active substance, the nature and amount of binder and the amount of talc and water, isopropanol or ethanol vary.

Other Examples are shown in Table 4.

TABLE 4

| | CGRP | | | | | | | | |
|---|---|---|---|---|---|---|---|---|---|
| | antagonist | (B) | *pbw | *pbw | *pbw | *pbw | | *pbw | *pbw |
| Ex. | (A) [*pbw] | [mg] | povidone | HPC | pellets | talc | *pbw isopropanol | EtOH | H₂O |
| 4.1 | 150 | 25.0 | 28.8 | 0.0 | 144.0 | 31.7 | 1890 | 0 | 0 |
| 4.2 | 210 | 2.5 | 24.3 | 0.0 | 121.5 | 26.7 | 1600 | 0 | 1600 |
| 4.3 | 250 | 2.8 | 24.4 | 0.0 | 121.8 | 26.8 | 0 | 1610 | 0 |
| 4.4 | 560 | 14.5 | 0.0 | 26.7 | 133.5 | 29.4 | 0 | 0 | 1760 |
| 4.5 | 450 | 2.5 | 0.0 | 24.3 | 121.5 | 26.7 | 0 | 1600 | 0 |
| 4.6 | 430 | 5.0 | 0.0 | 24.8 | 124.0 | 27.3 | 0 | 1640 | 0 |
| 4.7 | 70 | 10.0 | 25.8 | 0.0 | 129.0 | 28.4 | 1700 | 0 | 0 |
| 4.8 | 360 | 80.0 | 39.8 | 0.0 | 199.0 | 43.8 | 2630 | 0 | 0 |
| 4.9 | 530 | 100.0 | 0.0 | 43.8 | 219.0 | 48.2 | 0 | 2890 | 0 |
| 4.10 | 5 | 25.0 | 28.8 | 0.0 | 144.0 | 31.7 | 1900 | 0 | 0 |
| 4.11 | 230 | 50.0 | 33.8 | 0.0 | 169.0 | 37.2 | 2230 | 0 | 0 |
| 4.12 | 20 | 100.0 | 0.0 | 43.8 | 219.0 | 48.2 | 0 | 0 | 2890 |
| 4.13 | 230 | 90.0 | 0.0 | 41.8 | 209.0 | 46.0 | 4210 | 0 | 0 |
| 4.14 | 540 | 25.0 | 0.0 | 28.8 | 144.0 | 31.7 | 1900 | 0 | 0 |
| 4.15 | 500 | 50.0 | 0.0 | 33.8 | 169.0 | 37.2 | 2230 | 0 | 0 |
| 4.16 | 40 | 15.0 | 0.0 | 26.8 | 134.0 | 29.5 | 0 | 1800 | 0 |
| 4.17 | 150 | 85.0 | 0.0 | 40.8 | 204.0 | 44.9 | 0 | 0 | 4950 |
| 4.18 | 10 | 75.0 | 38.8 | 0.0 | 194.0 | 42.7 | 2600 | 0 | 0 |
| 4.19 | 140 | 100.0 | 43.8 | 0.0 | 219.0 | 48.2 | 3610 | 0 | 0 |
| 4.20 | 430 | 80.0 | 0.0 | 39.8 | 199.0 | 43.8 | 0 | 0 | 2670 |
| 4.21 | 250 | 20.0 | 0.0 | 27.8 | 139.0 | 30.6 | 1870 | 0 | 0 |
| 4.22 | 120 | 80.0 | 0.0 | 39.8 | 199.0 | 43.8 | 2670 | 0 | 0 |
| 4.23 | 260 | 12.7 | 0.0 | 26.3 | 131.7 | 29.0 | 0 | 0 | 1770 |
| 4.24 | 500 | 25.0 | 28.8 | 0.0 | 144.0 | 31.7 | 0 | 0 | 1930 |
| 4.25 | 1000 | 5.9 | 25.0 | 0.0 | 124.9 | 27.5 | 0 | 1680 | 0 |
| 4.26 | 400 | 60.0 | 35.8 | 0.0 | 179.0 | 39.4 | 0 | 2400 | 0 |
| 4.27 | 440 | 75.0 | 38.8 | 0.0 | 194.0 | 42.7 | 0 | 3210 | 0 |
| 4.28 | 350 | 25.0 | 0.0 | 28.8 | 144.0 | 31.7 | 0 | 0 | 1930 |
| 4.29 | 210 | 100.0 | 0.0 | 43.8 | 219.0 | 48.2 | 0 | 0 | 2940 |

*pbw = parts by weight;
EtOH = ethanol

Extrudates

The medicaments according to the invention may also be prepared in the form of extrudates which after cutting/spheronising are packed directly into capsules or ground up and processed to form tablets.

The preparation method comprises the following steps:
1. Extrusion
2a. Cutting/Spheronising
2b. Grinding and subsequent processing into tablets

### EXAMPLE 5a

Preparation of Moist Extrudates with 300 Parts by Weight of CGRP Antagonist (A) or an Equivalent Amount of a Physiologically Acceptable Salt Thereof and 80 Parts by Weight of Serotonin Reuptake Inhibitor (B) or an Equivalent Amount of a Physiologically Acceptable Salt Thereof

Composition:

| | |
|---|---|
| povidone | 6 parts by weight |
| microcrystalline cellulose | 40 parts by weight |
| CGRP antagonist (A) | 300 parts by weight |
| serotonin reuptake inhibitor (B) | 80 parts by weight |

300 parts by weight CGRP antagonist (A), 80 parts by weight serotonin reuptake inhibitor (B), 40 parts by weight microcrystalline cellulose (Avicel PH 101) and 6 parts by weight povidone (collidone K25) are mixed for 15 minutes in a gyrowheel mixer. Then the powder mixture is placed in a twin-screw extruder together with water, at a speed of approx. 1 kg/h. The addition of water is automatically regulated to produce a torque of approx. 19% in the extruder. Extrusion is carried out through a nozzle plate with bores 0.8 mm in diameter.

The extruded strips are formed into rounded pellets in a Spheronizer, taking approx. 3 minutes at approx. 850 RPM.

The pellets are dried at 80° C. for approx. 1.5 h in a fluidised bed dryer.

The core material is fractionated through a tumbler screening machine with different sieve plates with nominal mesh sizes of from 0.71 to 1.25 mm. The appropriate product fractions between 0.71 and 0.90 and between 0.90 and 1.12 mm are used.

Other Examples are shown in Table 5.

TABLE 5

| | | serotonin | | | |
|---|---|---|---|---|---|
| | CGRP | reuptake | *pbw | | *pbw |
| | antagonist | inhibitor (B) | microcryst. | *pbw | polyethyleneglycol |
| Example | (A) [*pbw] | [*pbw] | cellulose | povidone | 4000 |
| 5.1 | 20 | 10 | 6.0 | 0.9 | 50 |
| 5.2 | 370 | 25 | 79.0 | 11.9 | |
| 5.3 | 160 | 20 | 36.0 | 5.4 | 144 |
| 5.4 | 110 | 2.5 | 22.5 | 3.4 | 90 |
| 5.5 | 110 | 50 | 32.0 | 4.8 | 128 |
| 5.6 | 370 | 20 | 78.0 | 11.7 | 312 |
| 5.7 | 250 | 50 | 60.0 | 9.0 | |
| 5.8 | 370 | 50 | 84.0 | 12.6 | |
| 5.9 | 390 | 22.1 | 82.4 | 12.4 | |
| 5.10 | 40 | 200 | 48.0 | 7.2 | 192 |
| 5.11 | 200 | 10 | 42.0 | 6.3 | |
| 5.12 | 30 | 7.5 | 7.5 | 1.1 | 30 |
| 5.13 | 380 | 20 | 80.0 | 12.0 | |
| 5.14 | 360 | 20 | 76.0 | 11.4 | |
| 5.15 | 250 | 25 | 55.0 | 8.3 | |
| 5.16 | 160 | 22.1 | 36.4 | 5.5 | |
| 5.17 | 10 | 80 | 18.0 | 2.7 | 72 |
| 5.18 | 380 | 60 | 88.0 | 13.2 | |
| 5.19 | 160 | 60 | 44.0 | 6.6 | |
| 5.20 | 260 | 50 | 62.0 | 9.3 | |

*pbw = parts by weight

### EXAMPLE 6

Preparation of Molten Extrudates Containing 200 Parts by Weight CGRP Antagonist (A) or an Equivalent Amount of a Physiologically Acceptable Salt Thereof and 60 Parts by Weight Serotonin Reuptake Inhibitor (B) or an Equivalent Amount of a Physiologically Acceptable Salt Thereof

Composition:

| | |
|---|---|
| povidone | 6 parts by weight |
| Poloxamer | 40 parts by weight |
| CGRP antagonist (A) | 200 parts by weight |
| serotonin reuptake inhibitor (B) | 60 parts by weight |

200 parts by weight CGRP antagonist (A), 60 parts by weight serotonin reuptake inhibitor (B), 40 parts by weight poloxamer and 6 parts by weight povidone K25 are mixed for 15 minutes in a gyrowheel mixer. Then the powder mixture is placed in a twin-screw extruder at a speed of approx. 1 kg/h. The temperature is regulated to produce a torque of approx. 19% in the extruder. Extrusion is carried out through a nozzle plate with bores 0.8 mm in diameter.

The extruded strips are cut and formed into rounded pellets in a Spheronizer, taking approx. 3 minutes at approx. 850 RPM at about 40° C.

The pellets are dried at 80° C. for approx. 1.5 h in a fluidised bed dryer.

The core material is fractionated through a tumbler screening machine with different sieve plates with nominal mesh sizes of from 0.71 to 1.25 mm. The appropriate product fractions between 0.71 and 0.90 and between 0.90 and 1.12 mm are used in subsequent processes.

The compositions may vary and are shown in tabulated form below.

TABLE 6

| | | serotonin | | | |
|---|---|---|---|---|---|
| | CGRP | reuptake | | | *pbw |
| | antagonist (A) | inhibitor | *pbw | *pbw | polyethyleneglycol |
| Example | [*pbw] | (B) [*pbw] | povidone | poloxamer | 4000 |
| 6.1 | 140 | 60 | 3.0 | 50.8 | |
| 6.2 | 200 | 22.1 | 3.3 | 56.4 | |
| 6.3 | 330 | 25 | 5.3 | 90.1 | |
| 6.4 | 160 | 15 | 2.6 | 44.4 | |
| 6.5 | 10 | 50 | 0.9 | 15.2 | 45.7 |
| 6.6 | 230 | 2.5 | 3.5 | 59.0 | |
| 6.7 | 140 | 12.5 | 2.3 | 38.7 | |
| 6.8 | 70 | 60 | 2.0 | 33.0 | 99.0 |
| 6.9 | 390 | 10 | 6.0 | 101.5 | |
| 6.10 | 330 | 10 | 5.1 | 86.3 | |
| 6.11 | 380 | 2.5 | 5.7 | 97.1 | |
| 6.12 | 360 | 40 | 6.0 | 101.5 | |
| 6.13 | 270 | 25 | 4.4 | 74.9 | |
| 6.14 | 400 | 60 | 6.9 | 116.7 | |
| 6.15 | 280 | 75 | 5.3 | 90.1 | |
| 6.16 | 230 | 40 | 4.1 | 68.5 | |
| 6.17 | 120 | 60 | 2.7 | 45.7 | |
| 6.18 | 150 | 5 | 2.3 | 39.3 | |
| 6.19 | 180 | 100 | 4.2 | 71.1 | |
| 6.20 | 30 | 150 | 2.7 | 45.7 | 137.0 |
| 6.21 | 190 | 10 | 3.0 | 50.8 | |
| 6.22 | 390 | 50 | 6.6 | 111.7 | |
| 6.23 | 210 | 15 | 3.4 | 57.1 | |
| 6.24 | 60 | 10 | 1.1 | 17.8 | 53.3 |

*pbw = parts by weight

### EXAMPLE 7

Further Processing into Tablets

The extrudates are ground in a suitable mill, the resulting granules are further processed with conventional tabletting excipients analogously to Example 1 to form tablets.

Inhalable Powder

Preparation of Spherically Nanostructured Microparticles of the Active Substances for Preparing an Inhalable Powder

In order to prepare an active substance solution of 4 wt. % the active substances are dissolved accordingly in an ethanol/water (4:1) mixture and the active substance solution is sprayed so as to obtain a spray mist with a droplet size having the characteristic X50 (median value=particle size/droplet size, below which 50% of the quantity of particles falls relative to the distribution by volume of the individual particles/drops) in the range from 1.5 to 8 μm and Q(5.8) (corresponds to the quantity of particles, based on the distribution by volume of the droplets below 5.8 μm) of between 30 and 100% is obtained. The spray mist thus obtained is dried using a drying gas with an entry temperature of between 130° C. and 200° C. and an exit temperature of 40° C. to 120° C. The current by volume of the spray gas is 1 Nm³/h to 15 Nm³/h and the current by volume of the drying gas is 15 Nm³/h to 150 Nm³/h. The dried solid content is collected using a gravity precipitator and/or a filter unit.

### EXAMPLE 8

Capsules for Powder Inhalation Containing 0.5 mg CGRP Antagonist (A) or an Equivalent Amount of a Physiologically Acceptable Salt Thereof and 0.25 mg Serotonin Reuptake Inhibitor (B) or an Equivalent Amount of a Physiologically Acceptable Salt Thereof

Composition:
1 capsule for powder inhalation contains:

| | |
|---|---|
| CGRP antagonist (A) | 0.5 mg |
| serotonin reuptake inhibitor (B) | 0.25 mg |
| lactose | 20 mg |
| hard gelatine capsules | 50 mg |

Method of Preparation:

The active substance is prepared as spherically nanostructured particles of active substance and homogeneously mixed with lactose. The mixture is packed into hard gelatine capsules.

Other Examples are listed in Table 8.

TABLE 8

| | CGRP | serotonin reuptake | mg |
|---|---|---|---|
| Example | antagonist (A) [mg] | inhibitor (B) [mg] | lactose |
| 8.1 | 20.10 | 20.00 | 9.90 |
| 8.2 | 29.50 | 10.40 | 10.10 |
| 8.3 | 13.10 | 8.30 | 28.60 |
| 8.4 | 29.10 | 12.20 | 8.70 |
| 8.5 | 26.50 | 8.00 | 15.50 |
| 8.6 | 8.10 | 17.00 | 24.90 |
| 8.7 | 16.70 | 10.20 | 23.10 |
| 8.8 | 5.10 | 13.80 | 31.10 |
| 8.9 | 1.10 | 19.80 | 29.10 |
| 8.1 | 17.20 | 0.90 | 31.90 |
| 8.11 | 10.20 | 11.70 | 28.10 |
| 8.12 | 17.20 | 6.50 | 26.30 |
| 8.13 | 11.30 | 19.30 | 19.40 |
| 8.14 | 16.90 | 4.50 | 28.60 |
| 8.15 | 1.20 | 12.80 | 36.00 |
| 8.16 | 24.70 | 16.70 | 8.60 |
| 8.17 | 4.80 | 18.30 | 26.90 |
| 8.18 | 32.80 | 6.30 | 10.90 |
| 8.19 | 3.60 | 14.00 | 32.40 |
| 8.2 | 29.20 | 18.10 | 2.70 |
| 8.21 | 7.60 | 14.30 | 28.10 |
| 8.22 | 28.70 | 14.70 | 6.60 |
| 8.23 | 29.10 | 14.50 | 6.40 |
| 8.24 | 19.60 | 4.40 | 26.00 |
| 8.25 | 35.40 | 5.90 | 8.70 |
| 8.26 | 6.10 | 6.90 | 37.00 |
| 8.27 | 34.40 | 8.70 | 6.90 |

### EXAMPLE 9

Injectable Solution Containing 0.3 mg CGRP Antagonist (A) or an Equivalent Amount of a Physiologically Acceptable Salt Thereof and 0.2 mg Serotonin Reuptake Inhibitor (B) or an Equivalent Amount of a Physiologically Acceptable Salt Thereof

Composition:

| | |
|---|---|
| CGRP antagonist (A) | 0.3 mg |
| serotonin reuptake inhibitor (B) | 0.2 mg |
| physiological saline | |

The active substance is dissolved in physiological saline.

The amounts may vary and are shown in tabular form below.

TABLE 9

| | CGRP antagonist | serotonin reuptake |
|---|---|---|
| Example | (A) [pbw] | inhibitor (B) [pbw] |
| 9.1 | 0.20 | 0.07 |
| 9.2 | 14.30 | 4.77 |
| 9.3 | 4.40 | 1.47 |
| 9.4 | 10.30 | 3.43 |
| 9.5 | 1.80 | 0.60 |
| 9.6 | 50.00 | 16.67 |
| 9.7 | 4.40 | 1.47 |
| 9.8 | 9.40 | 3.13 |
| 9.9 | 2.60 | 0.87 |
| 9.10 | 8.20 | 2.73 |
| 9.11 | 4.30 | 1.43 |
| 9.12 | 25.50 | 8.50 |
| 9.13 | 14.20 | 4.73 |
| 9.14 | 13.40 | 4.47 |
| 9.15 | 5.40 | 1.80 |
| 9.16 | 6.90 | 2.30 |
| 9.17 | 7.70 | 2.57 |
| 9.18 | 30.00 | 10.00 |
| 9.19 | 8.30 | 2.77 |
| 9.20 | 13.10 | 4.37 |

### EXAMPLE 10

Suppositories Containing 200 mg CGRP Antagonist (A) or an Equivalent Amount of a Physiologically Acceptable Salt Thereof and 150 mg Serotonin Reuptake Inhibitor (B) or an Equivalent Amount of a Physiologically Acceptable Salt Thereof

Composition:

| | |
|---|---|
| CGRP antagonist (A) | 200 mg |
| serotonin reuptake inhibitor (B) | 150 mg |
| hard wax | ad 2 g |

Method of Preparation:

The hard wax is melted and the active substances are suspended in the mass. Then the mass is poured into suitable suppository moulds.

The amounts may vary and are shown in tabular form below.

TABLE 10

| | CGRP antagonist (A) | serotonin reuptake inhibitor |
|---|---|---|
| Example | [mg] | (B) [mg] |
| 10.1 | 250 | 20 |
| 10.2 | 280 | 10 |
| 10.3 | 460 | 60 |
| 10.4 | 540 | 70 |
| 10.5 | 320 | 120 |
| 10.6 | 180 | 10 |
| 10.7 | 150 | 180 |
| 10.8 | 480 | 160 |
| 10.9 | 590 | 40 |
| 10.10 | 180 | 170 |
| 10.11 | 520 | 40 |
| 10.12 | 540 | 100 |
| 10.13 | 110 | 90 |
| 10.14 | 560 | 140 |
| 10.15 | 50 | 120 |
| 10.16 | 320 | 150 |
| 10.17 | 440 | 30 |
| 10.18 | 590 | 20 |
| 10.19 | 140 | 180 |
| 10.20 | 340 | 110 |
| 10.21 | 180 | 30 |
| 10.22 | 140 | 190 |
| 10.23 | 260 | 160 |
| 10.24 | 340 | 50 |

1. A method for treating headaches, migraine and cluster headaches which comprises co-administering, to a host in need of such treatment, a CGRP-antagonist and a serotonin reuptake inhibitor, wherein the dosages of these two agents are selected so that they achieve a therapeutic effect when co-administered.

2. The method according to claim 1, wherein the CGRP antagonist is selected from the group consisting of:
(1) 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine,
(2) 1-[4-amino-3,5-dibromo-N-[[4-(2,3,4,5-tetrahydro-2(1H)-oxo-1,3-benzodiazepin-3-yl)-1-piperidinyl]carbonyl]-D-phenylalanyl]-4-(1-piperidinyl)-piperidine,
(3) 1-[N²-[4-amino-3,5-dibromo-N-[[4-(3,4-dihydro-2(1H)-oxoquinazolin-3-yl)-1-piperidinyl]carbonyl]-D-phenylalanyl]-L-lysyl]-4-(4-pyridinyl)-piperazine,
(4) 1-[N²-[4-amino-3,5-dibromo-N-[[4-(3,4-dihydro-2(1H)-oxoquinazolin-3-yl)-1-piperidinyl]carbonyl]-D-phenylalanyl]-L-lysyl]-4-(4-pyridinyl)-piperidine,
(5) 1-[N²-[3,5-dibromo-N-[[4-(1,3-dihydro-4-phenyl-2(2H)-oxoimidazol-1-yl)-1-piperidinyl]carbonyl]-D-tyrosyl]-L-lysyl]-4-(4-pyridinyl)-piperazine,
(6) 1-[N²-[4-amino-3,5-dibromo-N-[[4-(1,3-dihydro-4-phenyl-2(2H)-oxoimidazol-1-yl)-1-piperidinyl]carbonyl]-D-phenylalanyl]-L-lysyl]4-(4-pyridinyl)-piperazine,
(7) 1-[3,5-dibromo-N-[[4-(3,4-dihydro-2(1H)-oxothieno[3,4-d]pyrimidin-3-yl)-1-piperidinyl]carbonyl]-D-tyrosyl]4-(1-piperidinyl)-piperidine,
(8) 1-[4-amino-3,5-dibromo-N-[[4-(2,4-dihydro-5-phenyl-3(3H)-oxo-1,2,4-triazol-2-yl)-1-piperidinyl]carbonyl]-D-phenylalanyl]-4-(1-methyl-4-piperidinyl)-piperidine,
(9) 1-[4-amino-3,5-dibromo-N-[[4-(2,4-dihydro-5-phenyl-3(3H)-oxo-1,2,4-triazol-2-yl)-1-piperidinyl]carbonyl]-D-phenylalanyl]-4-(1-piperidinyl)-piperidine,
(10) 1-[4-amino-3,5-dibromo-N-[[4-(2,4-dihydro-5-phenyl-3(3H)-oxo-1,2,4-triazol-2-yl)-1-piperidinyl]carbonyl]-D-phenylalanyl]-4-(1-methyl-4-piperidinyl)-piperazine,
(11) 1-[4-amino-3,5-dibromo-N-[[4-(3,4-dihydro-2(1H)-oxothieno[3,2-d]pyrimidin-3-yl)-1-piperidinyl]carbonyl]-D-phenylalanyl]-4-(1-piperidinyl)-piperidine,
(12) 1-[4-amino-3,5-dibromo-N-[[4-[1,3-dihydro-4-[3-(trifluoromethyl)phenyl]-2(2H)-oxoimidazol-1-yl]-1-piperidinyl]carbonyl]-D-phenylalanyl]-4-(1-ethyl-4-piperidinyl)-piperidine,
(13) 1-[4-amino-3,5-dibromo-N-[[4-[3,4-dihydro-2(1H)-oxoquinazolin-3-yl]-1-piperidinyl]carbonyl]-D-phenylalanyl]-4-(1-hexyl-4-piperidinyl)-piperidine,
(14) 1-[4-amino-3,5-dibromo-N-[[4-[3,4-dihydro-2(1H)-oxoquinazolin-3-yl]-1-piperidinyl]carbonyl]-D-phenylalanyl]-4-(1-cyclopropylmethyl-4-piperidinyl)-piperidine,
(15) 1-[N-[[4-[3,4-dihydro-2(1H)-oxoquinazolin-3-yl]-1-piperidinyl]carbonyl]-3-ethenyl-D, L-phenylalanyl]4-(hexahydro-1H-1-azepinyl)-piperidine,
(16) (R,S)-1-[4-[4-(3,4-dihydro-2(1H)-oxoquinazolin-3-yl)-1-piperidinyl]-2-[(4-hydroxy-3,5-dimethylphenyl)methyl]-1,4-dioxobutyl]4-(1-piperidinyl)-piperidine,
(17) 1-[4-amino-3,5-dibromo-N-[[4-[N-(aminocarbonyl)-N-phenylamino]-1-piperidinyl]carbonyl]-D-phenylalanyl]-4-(1-piperidinyl)-piperidine,
(18) 1-[4-amino-3,5-dibromo-N-[[4-(3,4-dihydro-2(1H)-oxoquinazolin-3-yl)-1-piperidinyl]carbonyl]-D-phenylalanyl]-4-(5-methoxy-4-pyrimidinyl)-piperazine,
(19) 1-[4-amino-3,5-dibromo-N-[[4-(1,1-dioxido-3(4H)-oxo-1,2,4-benzothiadiazin-2-yl)-1-piperidinyl]carbonyl]-D-phenylalanyl]-4-(1-piperidinyl)-piperidine,
(20) 1-[4-amino-3,5-dibromo-N-[[4-[2(1H)-oxoquinolin-3-yl]-1-piperidinyl]carbonyl]-D-phenylalanyl]-4-(1-piperidinyl)-piperidine,
(21) 1-[4-amino-3,5-dibromo-N-[[4-[3,4-dihydro-2(1H)-oxoquinazolin-3-yl]-1-piperidinyl]carbonyl]-D-phenylalanyl]-4-[3-(dimethylamino)propyl]-piperazine,
(22) 1-[4-amino-3,5-dibromo-N-[[4-[3,4-dihydro-2(1H)-oxoquinazolin-3-yl]-1-piperidinyl]carbonyl]-D-phenylalanyl]-4-(4-methyl-1-piperazinyl)-piperidine,
(23) 1-[4-amino-3,5-dibromo-N-[[4-[3,4-dihydro-2(1H)-oxoquinazolin-3-yl]-1-piperidinyl]carbonyl]-D-phenylalanyl]-4-[(1-methyl-4-piperidinyl)carbonyl]-piperazine,
(24) 1-[4-amino-3,5-dibromo-N-[[4-[3,4-dihydro-2(1H)-oxoquinazolin-3-yl]-1-piperidinyl]carbonyl]-D-phenylalanyl]-4-[(1-methyl-4-piperazinyl)carbonyl]-piperazine,
(25) 1-[4-amino-3,5-dibromo-N-[[4-(3,4-dihydro-2(1H)-oxoquinazolin-3-yl)-1-piperidinyl]carbonyl]-D-phenylalanyl]-4-[4-[4-(dimethylamino)butyl]phenyl]-piperazine,
(26) 1-[4-amino-3,5-dibromo-N-[[4-(3,4-dihydro-2(1H)-oxoquinazolin-3-yl)-1-piperidinyl]carbonyl]-D-phenylalanyl]-4-[4-(dimethylamino)-1-piperidinyl]-piperidine,
(27) 1-[N²-[[4-(1,3-dihydro-4-phenyl-2(2H)-oxoimidazol-1-yl)-1-piperidinyl]carbonyl]-N′-methyl-D-tryptyl]-4-(4-methyl-1-piperazinyl)-piperidine,
(28) 1-[N²-[[4-(1,3-dihydro-4-phenyl-2(2H)-oxoimidazol-1-yl)-1-piperidinyl]carbonyl]-N′-(1,1-dimethylethoxycarbonyl)-D-tryptyl]-4-(1-methyl-4-piperidinyl)-piperidine,
(29) (R,S)-1-[4-[4-(3,4-dihydro-2(1H)-oxoquinazolin-3-yl)-1-piperidinyl]-2-[(3,5-di-bromo-4-methyl phenyl)methyl]-1,4-dioxobutyl]-4-(4-methyl-1-piperazinyl)-piperidine,
(30) (R,S)-1-[4-[4-(3,4-dihydro-2(1H)-oxoquinazolin-3-yl)-1-piperidinyl]-2-[(3,5-di-bromo-4-methoxyphenyl)methyl]-1,4-dioxobutyl]-4-(1-methyl-4-piperidinyl)-piperidine,
(31) (R,S)-1-[4-[4-(3,4-dihydro-2(1H)-oxoquinazolin-3-yl)-1-piperidinyl]-2-[(3,4-di-bromophenyl)methyl]-1,4-dioxobutyl]-4-(4-methyl-1-piperazinyl)-piperidine,
(32) 1-[N²-[N-[[4-(1,3-dihydro-2(2H)-oxobenzimidazol-1-yl)-1-piperidinyl]carbonyl]-3,5-dibromo-D-tyrosyl]-L-lysyl]-4-(4-pyridinyl)-piperazine,
(33) 1-[4-amino-3,5-dibromo-N-[[4-(1,3-dihydro-6-hydroxy-2(2H)-oxobenzimidazol-1-yl)-1-piperidinyl]carbonyl]-D-phenylalanyl]-4-(1-piperidinyl)-piperidine,
(34) 1-[N²-[4-amino-3,5-dibromo-N-[[4-(1,3-dihydro-2(2H)-oxobenzimidazol-1-yl)-1-piperidinyl]carbonyl]-D-phenylalanyl]-N⁶,N⁶-dimethyl-L-lysyl]-4-(4-pyridinyl)-piperazine,
(35) 1-[N²-[4-amino-3,5-dibromo-N-[[4-(3,4-dihydro-2(1H)-oxoquinazolin-3-yl)-1-piperidinyl]carbonyl]-D-phenylalanyl]-N⁶,N⁶-dimethyl-L-lysyl]-4-(4-pyridinyl)-piperazine,
(36) (R,S)-1-[2-(4-amino-3,5-dibromobenzoyl)-4-[4-(3,4-dihydro-2(1H)-oxo-quinazolin-3-yl)-1-piperidinyl]-4-oxobutyl]-4-(1-piperidinyl)-piperidine,
(37) 1-[4-amino-3,5-dibromo-N-[[4-(3,4-dihydro-2,2-dioxido-2,1,3-benzothiadiazin-3-yl)-1-piperidinyl]carbonyl]-D-phenylalanyl]-4-(1-piperidinyl)-piperidine,
(38) 1-[4-amino-3,5-dibromo-N-[[4-[1,3-dihydro-2(2H)-oxoimidazo[4,5-c]quinolin-3-yl]-1-piperidinyl]carbonyl]-D-phenylalanyl]-4-(1-piperidinyl)carbonyl]-piperidine,
(39) 1-[4-amino-3,5-dibromo-N-[[4-(3,4-dihydro-2(1H)-oxoquinazolin-3-yl)-1-piperidinyl]carbonyl]-D-phenylalanyl]-4-(1-piperidinyl)-piperidine,
(40) 1-[N²-[3,5-dibromo-N-[[4-(3,4-dihydro-2(1-oxoquinazolin-3-yl)-1-piperidinyl]carbonyl]-D-tyrosyl]-N⁶,N⁶-dimethyl-L-lysyl]-4-(4-pyridinyl)-piperazine,
(41) 1-[4-amino-N-[[4-[4-(3-bromophenyl)-1,3-dihydro-2(2H)-oxoimidazol-1-yl]-1-piperidinyl]carbonyl]-3,5-dibromo-D-phenylalanyl]-4-(1-methyl-4-piperidinyl)-piperidine,
(42) 1-[4-amino-3,5-dibromo-N-[[4-[1,3-dihydro-4-phenyl-2(2H)-oxoimidazol-1-yl]-1-piperidinyl]carbonyl]-D-phenylalanyl]-4-(4-methyl-1-piperazinyl)-piperidine,
(43) 1-[4-amino-3,5-dibromo-N-[[4-[1,3-dihydro-4-[3-(trifluoromethyl)phenyl]-2(2H)-oxoimidazol-1-yl]-1-piperidinyl]carbonyl]-D-phenylalanyl]-4-(1-piperidinyl)-piperidine,
(44) 1-[4-amino-3,5-dibromo-N-[[4-[1,3-dihydro-4-[3-(trifluoromethyl)phenyl]-2(2H)-oxoimidazol-1-yl]-1-piperidinyl]carbonyl]-D-phenylalanyl]-4-(exo-8-methyl-8-azabicyclo[3.2.1]oct-3-yl)-piperazine,
(45) 1-[3,5-dibromo-N-[[4-(1,3-dihydro-4-phenyl-2(2H)-oxoimidazol-1-yl)-1-piperidinyl]carbonyl]-D-tyrosyl]-4-(1-piperidinyl)-piperidine,
(46) 1-[4-amino-3,5-dibromo-N-[[4-[1,3-dihydro-4-[3-(trifluoromethyl)phenyl]-2(2H)-oxoimidazol-1-yl]-1-piperidinyl]carbonyl]-D-phenylalanyl]-4-(1-ethyl-4-piperidinyl)-piperazine,
(47) 1-[4-amino-3,5-dibromo-N-[[4-[1,3-dihydro-4-phenyl-2(2H)-oxoimidazol-1-yl]-1-piperidinyl]carbonyl]-D-phenylalanyl]-4-(hexahydro-4-methyl-1H-1,4-diazepin-1-yl)piperidine,
(48) 1-[3,5-dibromo-N-[[4-(3,4-dihydro-2(1H)-oxoquinazolin-3-yl)-1-piperidinyl]-carbonyl]-D-tyrosyl]4-[1-(methylsulphonyl)-4-piperidinyl]-piperidine,
(49) 1-[4-amino-3,5-dibromo-N-[[4-(1,3-dihydro-4-phenyl-2(2H)-oxoimidazol-1-yl)-1-piperidinyl]carbonyl]-D-phenylalanyl]-4-(1-methyl-4-piperidinyl)-piperidine,
(50) 1-[3,5-dibromo-N-[[4-[1,3-dihydro-4-[3-(trifluoromethyl)phenyl]-2(2H)-oxo-imidazol-1-yl]-1-piperidinyl]carbonyl]-D-tyrosyl]-4-(hexahydro-1H-1-azepinyl)-piperidine,
(51) 1-[3,5-dibromo-N-[[4-(3,4-dihydro-2(1H)-oxoquinazolin-3-yl)-1-piperidinyl]-carbonyl]-D-tyrosyl]-4-(1-methyl-4-piperidinyl)-piperidine,
(52) 1-[4-amino-3,5-dibromo-N-[[4-(3,4-dihydro-2(1H)-oxoquinazolin-3-yl)-1-piperidinyl]carbonyl]-D-phenylalanyl]-4-(exo-8-methyl-8-azabicyclo[3.2.1]oct-3-yl)-piperazine,
(53) 1-[4-amino-3,5-dibromo-N-[[4-(1,3-dihydro-4-phenyl-2(2H)-oxoimidazol-1-yl)-1-piperidinyl]carbonyl]-D-phenylalanyl]-4-(1-methyl-4-piperidinyl)-piperazine,
(54) 1-[3,5-dibromo-N-[[4-[1,3-dihydro-4-[3-(trifluoromethyl)phenyl]-2(2H)-oxo-imidazol-1-yl]-1-piperidinyl]carbonyl]-D-tyrosyl]-4-(1-piperidinyl)-piperidine,
(55) 1-[N⁶-acetyl-N²-[3,5-dibromo-N-[[4-(3,4-dihydro-2(1-oxoquinazolin-3-yl)-1-piperidinyl]carbonyl]-D-tyrosyl]-L-lysyl]-4-(4-pyridinyl)-piperazine,
(56) 1-[3,5-dibromo-N-[[4-(1,3-dihydro-4-phenyl-2(2H)-oxoimidazol-1-yl)-1-piperidinyl]carbonyl]-D-tyrosyl]-4-(hexahydro-1H-1-azepinyl)-piperidine,
(57) 1-[4-amino-3,5-dibromo-N-[[4-(1,3-dihydro-4-(3-thienyl)-2(2H)-oxoimidazol-1-yl)-1-piperidinyl]carbonyl]-D-phenylalanyl]-4-(1-methyl-4-piperidinyl)-piperidine,
(58) 1-[4-amino-3,5-dibromo-N-[[4-[1,3-dihydro-4-[3-(trifluoromethyl)phenyl]-2(2H)-oxoimidazol-1-yl]-1-piperidinyl]carbonyl]-D-phenylalanyl]4-(1-methyl-4-piperidinyl)-piperidine,
(59) 1-[3,5-dibromo-N-[[4-(3,4-dihydro-2(1H)-oxoquinazolin-3-yl)-1-piperidinyl]carbonyl]-D-tyrosyl]-4-[1-(hydroxycarbonylmethyl)-4-piperidinyl]-piperidine,
(60) 1-[4-amino-3,5-dibromo-N-[[4-[3,4-dihydro-2(1H)-oxoquinazolin-3-yl]-1-piperidinyl]carbonyl]-D-phenylalanyl]-4-(1-methylsulphonyl-4-piperidinyl)-piperidine,
(61) 1-[3,5-dibromo-N-[[4-(3,4-dihydro-2(1H)-oxoquinazolin-3-yl)-1-piperidinyl]carbonyl]-D-tyrosyl]-4-(4-piperidinyl)-piperidine,
(62) 1-[4-amino-3,5-dibromo-N-[[4-(1,3-dihydro-4-phenyl-2(2H)-oxoimidazol-1-yl)-1-piperidinyl]carbonyl]-D-phenylalanyl]-4-(1-ethyl-4-piperidinyl)-piperidine,
(63) 1-[4-amino-3,5-dibromo-N-[[4-(1,3-dihydro-4-(3-hydroxyphenyl)-2(2H)-oxo-imidazol-1-yl)-1-piperidinyl]carbonyl]-D-phenylalanyl]-4-(1-methyl-4-piperidinyl)-piperidine,
(64) 1-[3,5-dibromo-N-[[4-(3,4-dihydro-2(1H)-oxoquinazolin-3-yl)-1-piperidinyl]carbonyl]-D-tyrosyl]-4-(hexahydro-1H-1-azepinyl)-piperidine,
(65) 1-[4-amino-3,5-dibromo-N-[[4-(1,3-dihydro-4-phenyl-2(2H)-oxoimidazol-1-yl)-1-piperidinyl]carbonyl]-D-phenylalanyl]-4-(1-piperidinyl)-piperidine,
(66) 1-[4-amino-3,5-dibromo-N-[[4-[4-(3-bromophenyl)-1,3-dihydro-2(2H)-oxo-imidazol-1-yl]-1-piperidinyl]carbonyl]-D-phenylalanyl]-4-(exo-8-methyl-8-azabicyclo[3.2.1]oct-3-yl)-piperazine,
(67) 1-[4-amino-3,5-dibromo-N-[[4-(3,4-dihydro-2(1H)-oxoquinazolin-3-yl)-1-piperidinyl]carbonyl]-D-phenylalanyl]-4-(1-ethyl-4-piperidinyl)-piperidine,
(68) 1-[4-amino-3,5-dibromo-N-[[4-(3,4-dihydro-2(1H)-oxoquinazolin-3-yl)-1-piperidinyl]carbonyl]-D-phenylalanyl]-4-(1-ethyl-4-piperidinyl)-piperazine,
(69) 1-[4-amino-3,5-dibromo-N-[[4-[1,3-dihydro-4-(3-methoxyphenyl)-2(2H)-oxo-imidazol-1-yl]-1-piperidinyl]carbonyl]-D-phenylalanyl]-4-(exo-8-methyl-8-azabicyclo[3.2.1]oct-3-yl)-piperazine,
(70) 1-[3,5-dibromo-N-[[4-(3,4-dihydro-2(1H)-oxoquinazolin-3-yl)-1-piperidinyl]carbonyl]-D-tyrosyl]-4-[1-(cyclopropylmethyl)-4-piperidinyl]-piperidine,
(71) 1-[4-amino-3,5-dibromo-N-[[4-(3,4-dihydro-2(1H)-oxoquinazolin-3-yl)-1-piperidinyl]carbonyl]-D-phenylalanyl]-4-(hexahydro-1H-1-azepinyl)-piperidine,
(72) 1-[4-amino-3,5-dibromo-N-[[4-(3,4-dihydro-2(1H)-oxoquinazolin-3-yl)-1-piperidinyl]carbonyl]-D-phenylalanyl]-4-(4-piperidinyl)-piperidine,
(73) 1-[3,5-dibromo-N-[[4-(1,3-dihydro-4-phenyl-2(2H)-oxoimidazol-1-yl)-1-piperidinyl]carbonyl]-D-tyrosyl]-4-(4-pyridinyl)-piperidine,
(74) 1-[3,5-dibromo-N-[[4-[1,3-dihydro-4-[3-(trifluoromethyl)phenyl]-2(2H)-oxo-imidazol-1-yl]-1-piperidinyl]carbonyl]-D-tyrosyl]-4-(1-methyl-4-piperidinyl)-piperazine,
(75) 1-[N²-[3,5-dibromo-N-[[4-[1,3-dihydro-4-[3-(trifluoromethyl)phenyl]-2(2H)-oxoimidazol-1-yl]-1-piperidinyl]carbonyl]-D-tyrosyl]-L-lysyl]-4-(4-pyridinyl)-piperazine,
(76) 1-[3,5-dibromo-N-[[4-(1,3-dihydro-4-(3-thienyl)-2(2H)-oxoimidazol-1-yl)-1-piperidinyl]carbonyl]-D-tyrosyl]-4-(1-piperidinyl)-piperidine,
(77) 1-[4-amino-N-[[4-[4-(3-chlorophenyl)-1,3-dihydro-2(2H)-oxoimidazol-1-yl]-1-piperidinyl]carbonyl]-3,5-dibromo-D-phenylalanyl]-4-(1-methyl-4-piperidinyl)-piperidine,
(78) 1-[4-amino-3,5-dibromo-N-[[4-[1,3-dihydro-4-[3-(trifluoromethyl)phenyl]-2(2H)-oxoimidazol-1-yl]-1-piperidinyl]carbonyl]-D-phenylalanyl]-4-(hexahydro-1H-1-azepinyl)-piperidine,
(79) 1-[4-amino-3,5-dibromo-N-[[4-[1,3-dihydro-4-[3-(trifluoromethyl)phenyl]-2(2H)-oxoimidazol-1-yl]-1-piperidinyl]carbonyl]-D-phenylalanyl]-4-(1-methyl-4-piperidinyl)-piperazine,
(80) 1-[4-amino-N-[[4-[4-(3-chlorophenyl)-1,3-dihydro-2(2H)-oxoimidazol-1-yl]-1-piperidinyl]carbonyl]-3,5-dibromo-D-phenylalanyl]-4-(hexahydro-1H-1-azepinyl)-piperidine,
(81) 1-[4-amino-3,5-dibromo-N-[[4-(1,3-dihydro-4-phenyl-2(2H)-oxoimidazol-1-yl)-1-piperidinyl]carbonyl]-D-phenylalanyl]-4-(4-pyridinyl)-piperazine,
(82) 1-[3,5-dibromo-N-[[4-(1,3-dihydro-4-phenyl-2(2H)-oxoimidazol-1-yl)-1-piperidinyl]carbonyl]-D-tyrosyl]-4-(1-methyl-4-piperidinyl)-piperidine,
(83) 1-[4-amino-3,5-dibromo-N-[[4-[1,3-dihydro-4-phenyl-2(2H)-oxoimidazol-1-yl]-1-piperidinyl]carbonyl]-D-phenylalanyl]-4-[4-(1-oxoethyl)phenyl]-piperazine,
(84) 1-[3,5-dibromo-N-[[4-[3,4-dihydro-2(1H)-oxoquinazolin-3-yl]-1-piperidinyl]carbonyl]-D-tyrosyl]-4-(1-methyl-4-piperidinyl)-piperazine,
(85) 1-[4-amino-3,5-dibromo-N-[[4-[1,3-dihydro-4-(3-nitrophenyl)-2(2H)-oxoimidazol-1-yl]-1-piperidinyl]carbonyl]-D-phenylalanyl]-4-(1-methyl-4-piperidinyl)-piperidine,
(86) 1-[4-amino-3,5-dibromo-N-[[4-[3,4-dihydro-2(1-oxoquinazolin-3-yl]-1-piperidinyl]carbonyl]-D-phenylalanyl]-4-(1-pyrrolidinyl)-piperidine,
(87) 1-[4-amino-3,5-dibromo-N-[[4-(1,3-dihydro-4-phenyl-2(2H)-oxoimidazol-1-yl)-1-piperidinyl]carbonyl]-D-phenylalanyl]-4-(hexahydro-1H-1-azepinyl)-piperidine and
(88) 1-[4-amino-3,5-dibromo-N-[[4-(1,3-dihydro-4-(3-thienyl)-2(2H)-oxoimidazol-1-yl)-1-piperidinyl]carbonyl]-D-phenylalanyl]-4-(1-methyl-4-piperidinyl)-piperazine,
or is a physiologically acceptable salt thereof.

3. The method according to claim 1 wherein the serotonin reuptake inhibitor is selected from the group consisting of citalopram, duloxetine, escitalopram, fluoxetine, fluvoxamine, paroxetine, sertraline and trazodone or is a physiologically acceptable salt thereof.

4. The method according to claim 3, wherein the serotonin reuptake inhibitor is duloxetine or a physiologically acceptable salt thereof.

5. The method according to claim 1, wherein the selected CGRP antagonist is administered by the intravenous or subcutaneous route in a dosage of 0.0001 to 3 mg/kg body weight, by the oral route in a dosage of 0.1 to 20 mg/kg body weight or by the nasal or inhalative route in a dosage of 0.1 to 10 mg/kg body weight once, twice or three times a day and
the serotonin reuptake inhibitor is administered by the oral route in a dosage of 0.03 to 1.43 mg/kg body weight once, twice or three times a day or
by the intravenous or subcutaneous route in a dosage of 0.002 to 0.09 mg/kg body weight once or twice a day or
by the rectal route in a dosage of 0.007 to 0.36 mg/kg body weight once or twice a day or
by the nasal route in a dosage of 0.006 to 0.29 mg/kg body weight once or twice a day.

6. A pharmaceutical composition comprising a CGRP antagonist and a serotonin reuptake inhibitor.

7. A pharmaceutical composition according to claim 6, comprising, in a single dosage unit, 0.1 to 1500 mg of a CGRP antagonist and,
in a single dosage unit, 0.1 to 150 mg of a serotonin reuptake inhibitor.

8. A kit of parts comprising:
(a) a first enclosure containing a pharmaceutical composition comprising a therapeutically effective amount of a CGRP antagonist; and
(b) a second enclosure containing a pharmaceutical composition comprising a serotonin reuptake inhibitor.

9. A kit of parts according to claim 8, the kit containing duloxetine or a physiologically acceptable salt thereof in the second enclosure.
